(19) United States
(12) Patent Application Publication
(10) Pub. No.: US 2012/0265300 A1
(43) Pub. Date: Oct. 18, 2012

(54) Aligned Fibrous Materials With Spatially Varying Fiber Orientation and Related Methods

(75) Inventors: Robert L. Mauck, Philadelphia, PA (US); Matthew B. Fisher, Media, PA (US); Brendon M. Baker, Philadelphia, PA (US); Amy M. Silverstein, Philadelphia, PA (US); Jason Alan Burdick, Philadelphia, PA (US)

(73) Assignee: THE TRUSTEES OF THE UNIVERSITY OF PENNSYLVANIA, Philadelphia, PA (US)

(21) Appl. No.: 13/422,027

(22) Filed: Mar. 16, 2012

Related U.S. Application Data
(63) Continuation-in-part of application No. PCT/US2010/049111, filed on Sep. 16, 2010.
(60) Provisional application No. 61/243,660, filed on Sep. 18, 2009.
(60) Provisional application No. 61/594,551, filed on Feb. 3, 2012.

(51) Int. Cl. B32B 5/16; B32B 27/02; D02G 3/22; A61K 35/00; B32B 37/14; B29C 47/00; A61K 9/00; B32B 5/12; A61F 2/38

(52) U.S. Cl. 623/14.12; 428/323; 428/113; 428/411.1; 428/338; 428/114; 156/275.5; 264/465; 424/400; 424/93.1

(57) ABSTRACT

Provided are materials comprising layers of anisotropically aligned fibers, the alignment of which fibers may be adjusted so as to give rise to circumferentially-aligned fibers that replicate the fiber alignment of native fibrous tissue, such as the meniscus or the annulus fibrosis. Also provided are laminates formed from the disclosed materials, as well as methods of fabricating the disclosed materials and laminates.

### RELATED APPLICATIONS

The present application is a continuation-in-part of international application PCT/US2010/049111, “Artificial Meniscal Implants,” filed on Sep. 16, 2010, which international application claims priority to U.S. Application 61/243,660, “Artificial Meniscal Implants,” filed on Sep. 18, 2009. The present application also claims priority to U.S. Application 61/594,551, “Aligned Fibrous Materials With Spatially Varying Fiber Orientation And Related Methods,” filed on Feb. 3, 2012. All of the foregoing applications are incorporated herein by reference in their entireties for any and all purposes.

### GOVERNMENT RIGHTS

This work was supported by the National Institutes of Health (grant no. R01 AR056624) and by the Department of Veterans Affairs (grant no. I01RX000174). The government has rights in this invention.

### TECHNICAL FIELD

The present disclosure relates to the fields of biocompatible implant materials and to biocompatible polymer fibers.

### BACKGROUND

The menisci are crescent-shaped fibrocartilaginous tissues that function to transmit and distribute loads between the femur and tibia of the knee joint. As such, the meniscus experiences complex loads, including tension, compression, and shear. Meniscus function in tension arises from an organized microstructure—bundles of highly aligned collagen circumnavigate the tissue between insertion sites on the tibial plateau. These aligned collagen bundles endow the tissue with mechanical properties that are highly anisotropic, and highest in the primary collagen orientation. Existing meniscus replacement materials, however, lack this unique structure and organization.

It is also known that load bearing fibrocartilaginous tissues of the musculoskeletal system, including the knee meniscus and the annulus fibrosus (AF) of the intervertebral disc, are prone to failure and have a limited reparative capacity once damaged. Both tissues are ordered hierarchical laminates: the knee meniscus has a preponderance of circumferential collagen bundles with interspersed, perpendicularly directed, ‘tie’ fibers, while the AF consists of multiple alternating layers of oriented (+/−about 30°) collagen fibers that form an angle-ply structure. The mechanical function of both tissues arises at least in part from this underlying fibrous architecture.

### SUMMARY

To address engineering the meniscus and other fibrous tissues, presented here are aligned nanofibrous scaffolds that can recapitulate this mechanical anisotropy. In natural tissues, fibers within the native tissue have a pronounced c-shaped, or otherwise angled, macroscopic organization. To replicate this macroscopic change in organization over the anatomic size of the meniscus, presented here is an electrospinning method that collects organized fibers on a spinning disc or other mandrel.

This disclosure also presents data concerning the structure and mechanics of nanofibrous scaffolds collected using this novel technique, as compared to compare to aligned scaffolds obtained from a traditional electrospinning approach. Without being bound to any particular theory, one may hypothesize that these circumferentially aligned (CircAl) scaffolds would behave similarly to linearly aligned (LinAl) scaffolds on short length scales, but exhibit marked differences in mechanics as the length scale increased.

This disclosure presents aligned nanofibrous scaffolds (formable from a variety of polymers) that can mimic the order of these native tissues, and direct cell and matrix organization with in vitro culture. Also disclosed are constructed biologic laminates, in which scaffold layers are fused with one another through cell mediated matrix-deposition with appositional culture. In some embodiments, the tensile characteristics of the scaffold may replicate those of a mammalian knee menisus. Since the materials of construction in some cases exhibit non-linear stress responses to strain and/or are biodegrade or bioerode when subjected to physiological fluids under physiological conditions, and the scaffold may continue to provide tensile support during this period of biodegradation or bioerosion over a range of strain conditions, it is often useful to characterize the scaffold in terms of these parameters. That is, in various embodiments, a material (e.g., a scaffold) according to the present disclosure exhibits an overall circumferential modulus that is in the range of about 10 MPa to about 200 MPa, preferably at least about 20 MPa, more preferably at least about 40 MPa, still more preferably at least about 60 MPa, and most preferably at least about 80 MPa, at a strain region of about 10%, and/or an overall circumferential modulus in the range of about 5 to about 60 MPa, preferably at least 10 MPa, more preferably at least 20 MPa, and most preferably about 30-35 MPa, at a strain region of about 3%, and these properties are either retained or developed when the scaffold is subjected to physiological implant conditions for time sufficient to allow cell infiltration and meniscal healing, during and after which the components of the matrix are dissolved, bioeroded, or biodegraded into the patient. Preferably the modulus of the scaffold, after exposure to physiological fluids under physiological conditions, retains at least about 60% of its value after 7 days, and more preferably at least about 50% of its value after 60 days. In order to retain these modulus levels this invention also provides that the scaffolds have correspondingly, proportionately higher initial values. Unless otherwise stated herein, any reference to a specific target modulus is intended to reflect an initial value (i.e., before biodegradation or bioerosion and the changes in mechanical properties that develop as cells infiltrate and deposit new, load-bearing extracellular matrix within the scaffold substance). It should be understood that any and all mechanical characterizations or properties of materials set forth in international application PCT/US2010/049111 may apply to the materials disclosed herein.

Clinical application of these materials may, in some cases, benefit from implantation of already formed acellular multi-lamellar constructs. A ‘spot-welding’ method has been previously described in which method individual layers are bound together through local scaffold melting brought on by contacting at least one of the layers with a heated probe. This approach creates stable bi-layers, but can cause compression of the construct with insertion of the heated probes or arrays of probes.

An example of spot-welded layers is shown in FIG. 7. That figure illustrates insertion of a heated probe into two adjacent layers so as to fuse them together. The number of spot welds (lower left of figure) affects the mechanical properties of the final material, but the macroscopic structure of the spot-welded layers (lower left of figure) is affected by insertion of the heated probe. Here is presented a new method for forming nanofibrous laminates using light responsive materials, which materials may be polymeric fibers having nanoscale bodies (e.g., gold nanorods) disposed within. The nanoscale bodies effect controlled levels of heat with exposure to near-infrared (IR) light, which in turn allows for fusion of layers without physical contact from a probe or other instrument. This lack of contact in turn allows for layer fusion without the disruption of the layers' underlying structure that may result from contacting the layers with thermal probes or other implements.

In one embodiment, the present disclosure provides laminates (which may, in some places, also be referred to as compositions), the laminates comprising a first layer comprising a first population of polymeric fibers, at least some of the first population of polymeric fibers comprising nanoscale bodies disposed within; and a second layer comprising a second population of polymeric fibers, the first and second layers being bonded together at one or more locations.

The present disclosure also provides methods, the methods comprising irradiating a first fibrous layer comprising a first population of polymeric fibers having a first population of nanoscale bodies disposed within, the irradiating being performed so as to bond at least a portion of the first layer to a second fibrous layer comprising a second population of polymeric fibers.

Also provided are methods, the methods comprising electrospinning, from a polymeric fluid, a first population of polymeric fibers onto a first rotating surface of a mandrel, the electrospinning being performed such that at least a portion of the first population of polymeric fibers is aligned on the first surface in an arcuate (which may be characterized, in some cases, as being circumferential) fashion.

Further provided are compositions, the compositions comprising a first layer comprising a first population of polymeric fibers, the first population of polymeric fibers having an anisotropic alignment that varies spatially within the layer.

Additionally provided are biocompatible implants, the implants comprising a quantity of a composition according to the present disclosure, the quantity of material being shaped to as to approximate at least a portion of a knee meniscus, an annulus fibrosis, or any combination thereof.

Further provided are methods, the methods comprising seeding a composition according to the present disclosure with a population of cells.

### BRIEF DESCRIPTION OF THE DRAWINGS

The summary, as well as the following detailed description, is further understood when read in conjunction with the appended drawings. For the purpose of illustrating the disclosure, there are shown in the drawings exemplary embodiments of the disclosure; however, the disclosure is not limited to the specific methods, compositions, and devices disclosed. In addition, the drawings are not necessarily drawn to scale. In the drawings:

FIG. 1 presents a schematic of nanofibrous scaffold containing AuNRs interspersed in PCL fibers (A). MSCs align on NRS (B) and SEMs show no difference between PCL (C) and NRS (D), scale bar: 10 μm. Average stress-strain profiles for PCL and NRS (E). A small decrease in both modulus (F) and yield stress (G) was noted in NRS compared to PCL scaffolds (n=6, *p<0.05).

FIG. 2 presents a schematic of laminate construction and testing (left) and quantification (right) of maximum interface strength for 1 and 2 welds produced through heated probe or IR laser exposure of NRS (n=6, *=p<0.05);

FIG. 3 presents A) Bright field images of fibers collected on slides (4×). B) Plot of mean fiber angle as a function of position from center of scaffold;

FIG. 4 presents fluorescent imaging of actin (green) and nuclei (blue) for MSCs seeded on linearly aligned (A) and circumferentially aligned (B) scaffolds (scale bar=100 μm);

FIG. 5 presents A) Schematic of specimens taken for tensile testing from circumferentially aligned (CA) mats. B) Modulus of CA and linearly aligned specimens with varying radii (3 cm, 5 cm), sample length (short, long) and region for strain analysis (center, edge). (*p<0.05 between short and long groups, +p<0.05 between scaffold region). C) Representative strain plots for LinAl and CircAl scaffolds with a central region strain of 3%;

FIG. 6 illustrates the annulus fibrosis and the meniscus, two fibrous tissues;

FIG. 7 illustrates an existing method of forming a multi-lamellar nanofibrous structure;

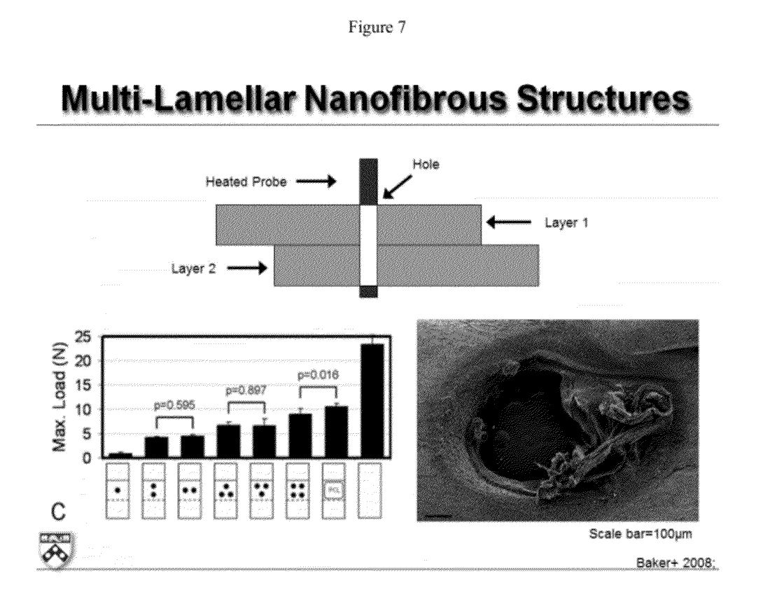

FIG. 8 illustrates a micrograph of an annulus fibrosis and also an image of a meniscus replacement material;

FIG. 9 illustrates exemplary parameters used in an experiment involving dispersion of gold nanorods in poly-caprolactone polymer;

FIG. 10 illustrates an exemplary process for fabricating multilamellar materials according to the present disclosure;

FIG. 11 illustrates exemplary results realized from fabricating multilamellar materials according to the present disclosure;

FIG. 12 illustrates caprolactone nanofiber morphology without (left) and with (right) inclusion of gold nanorods within the nanofibers;

FIG. 13 illustrates a less-magnified view of FIG. 12;

FIG. 14 illustrates the morphology of a fibrous material made from polycaprolactone fibers after spot welding (left) and nanorod-infrared welding (right);

FIG. 15 illustrates mechanical data obtained from testing fibrous scaffolds without (“PCL”) and with (“NRS”) nanorods;

FIG. 16 illustrates interface strength achieved by materials according to the present disclosure;

FIG. 17 presents cell viability and morphology results obtained on fibrous polycaprolactone scaffolds without (PCL) and with (NRS) nanorod incusion;

FIG. 18 depicts an exemplary method of fabricating anatomic meniscus structures according to the present disclosure; and

FIG. 19 depicts (A) a schematic showing electrospinning of fibers onto a rotating mandrel; (B) and (C) bright field images of strips of angled fibers with magnified images showing local alignment.

### DETAILED DESCRIPTION OF ILLUSTRATIVE EMBODIMENTS

The present disclosure may be understood more readily by reference to the following detailed description taken in connection with the accompanying figures and examples, which form a part of this disclosure. It is to be understood that this disclosure is not limited to the specific devices, methods, applications, conditions or parameters described and/or shown herein, and that the terminology used herein is for the purpose of describing particular embodiments by way of example only and is not intended to be limiting of the claims. Also, as used in the specification including the appended claims, the singular forms “a,” “an,” and “the” include the plural, and reference to a particular numerical value includes at least that particular value, unless the context clearly dictates otherwise.

The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable. Any documents cited herein are incorporated herein by reference in their entireties for any and all purposes. Further information may be found in U.S. patent applications 61/243,660 and 61/255,542, both of which are incorporated herein in their entireties for any and all purposes.

In a first embodiment, the present disclosure provides laminates. The laminates suitably include a first layer comprising a first population of polymeric fibers, with at least some of the first population of polymeric fibers comprising nanoscale bodies disposed within. The laminates suitably include a second layer comprising a second population of polymeric fibers, and the first and second layers are suitably bonded together at one or more locations.

The first population of polymeric fibers may comprise virtually any polymer. Polymers that are natural, synthetic, biocompatible, biodegradable, non-biodegradable, bioabsorbable, or any combination thereof are all suitable. It should be understood that in some embodiments, e.g., when an electrospun material is made of a single fiber (e.g. nanofiber), the fiber is folded thereupon, hence can be viewed as a plurality of connected fibers. It is to be understood that a more detailed reference to a plurality of fibers is not intended to limit the scope of the present disclosure to such particular case. Thus, unless otherwise defined, any reference herein to a “plurality of fibers” applies also to a single fiber and vice versa.

This disclosure is not limited by the thickness or shape of the fibers generated and used. Accordingly, the cross-sections of the fiber or fibers may be circular, oval, rectangular, square, or any shape which can be defined by the spinneret. Similarly, the fibers can have a cross-sectional dimension in the range of about 1 nm to about 10 microns, in the range of about 20 nm to about 1000 nm, in the range of about 100 nm to about 1000 nm, or in the range of about 1 micron to about 10 microns.

Fibers may be polymer fibers having diameters typically between 10 nm and 1000 nm. Exemplary sub-ranges contemplated by the present disclosure include between 100 and 1000 nm between 100 and 800 nm, between 100 and 600 nm, and between 100 and 400 nm. Other exemplary ranges include 10-100 nm, 10-200 nm and 10-500 nm. As mentioned, the fibers of the present disclosure are preferably generated by an electrospinning processes. In certain preferred embodiments, the first population of fibers, the second population of fibers, or both, has an average cross-sectional dimension in the range of from about 10 nm to about 10,000 nm.

As described herein, the various fibers may comprise materials that are natural, synthetic, biocompatible, biodegradable, non-biodegradable, and/or biosorbable, and unless specifically restricted to one or more of these categories, the fibers may comprise materials from any one of these categories.

The phrase “synthetic polymer” refers to polymers that are not found in nature, even if the polymers are made from naturally occurring biomaterials. Examples include, but are not limited to, aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polysiloxanes, and combinations thereof.

Suitable synthetic polymers for use according to the present disclosure may include biosynthetic polymers based on sequences found in collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides and combinations thereof.

The phrase “natural polymer” refers to polymers that are naturally occurring. Non-limiting examples of such polymers include, silk, collagen-based materials, chitosan, hyaluronic acid and alginate.

The phrase “biocompatible polymer” refers to any polymer (synthetic or natural) which when in contact with cells, tissues or body or physiological fluid of an organism does not induce adverse effects such as immunological reactions and/or rejections and the like. It will be appreciated that a biocompatible polymer can also be a biodegradable polymer.

The phrase “biodegradable polymer” refers to a synthetic or natural polymer which can be degraded (i.e., broken down) in the physiological environment such as by enzymes, microbes, or proteins. Biodegradability depends on the availability of degradation substrates (i.e., biological materials or portion thereof which are part of the polymer), the presence of biodegrading materials (e.g., microorganisms, enzymes, proteins) and the availability of oxygen (for aerobic organisms, microorganisms or portions thereof), carbon dioxide (for anaerobic organisms, microorganisms or portions thereof) and/or other nutrients. Aliphatic polyesters, poly(amino acids), polyalkylene oxalates, polyamides, polyamido esters, poly(anhydrides), poly(beta-amino esters), polycarbonates, polyethers, polyorthoesters, polyphosphazenes, and combinations thereof are considered biodegradable. More specific examples of biodegradable polymers include, but are not limited to, collagen (e.g., Collagen I or IV), fibrin, hyaluronic acid, polylactic acid (PLA), polyglycolic acid (PGA), polycaprolactone (PCL), poly(Lactide-co-Glycolide) (PLGA), polydioxanone (PDO), trimethylene carbonate (TMC), polyethyleneglycol (PEG), Collagen, PEG-DMA, alginate or alginic acid, chitosan polymers, or copolymers or mixtures thereof.

The phrase “non-biodegradable polymer” refers to a synthetic or natural polymer which is not degraded (i.e., broken down) in the physiological environment. Examples of non-biodegradable polymers include, but are not limited to, carbon, nylon, silicon, silk, polyurethanes, polycarbonates, polyacrylonitriles, polyanilines, polyvinyl carbazoles, polyvinyl chlorides, polyvinyl fluorides, polyvinyl imidazoles, polyvinyl alcohols, polystyrenes and poly(vinyl phenols), aliphatic polyesters, polyacrylates, polymethacrylates, acyl-sutostituted cellulose acetates, nonbiodegradable polyurethanes, polystyrenes, chlorosulphonated polyolefins, polyethylene oxides, polytetrafluoroethylenes, polydialkylsiloxanes, and shape-memory materials such as poly (styrene-block-butadiene), copolymers or mixtures thereof.

The phrase “biosorbable” refers to those polymers which are absorbed within the host body, either through a biodegradation process, or by simple dissolution in aqueous or other body fluids. Water soluble polymers, such as poly(ethylene oxide) are included in this class of polymers.

It will be appreciated that more than one polymer may be used to fabricate the scaffolds of the present disclosure. For example, the scaffold may be fabricated from a co-polymer. The term “co-polymer” as used herein, refers to a polymer of at least two chemically distinct monomers. Non-limiting examples of co-polymers which may be used to fabricate the scaffolds of the present disclosure include, PLA-PEG, PEGT-PBT, PLA-PGA, PEG-PCL and PCL-PLA. The use of copolymers or mixtures of polymers/copolymers provides a flexible means of providing the required blend of properties. In but one non-limiting example, functionalized poly(β-amino esters), which may be formed by the conjugate addition of primary or secondary amines with diacrylates, can provide a range of materials exhibiting a wide array of advantageous properties for this purpose. Such materials are described, for example, in Anderson, et al., “A Combinatorial Library of Photocrosslinkable and Degradable Materials,” Adv. Materials, vol. 18 (19), 2006, which reference is incorporated by reference in its entirety.

Additionally, individual polymers or co-polymers may be physically mixed and co-spun through the same spinneret. Similarly, according to this disclosure, a composition may be comprised of a mixture of simultaneously or sequentially delivered polymers and/or copolymers. This includes mixtures of at least two natural, synthetic, biocompatible, biodegradable, non-biodegradable, and/or biosorbable polymers and co-polymers.

Other embodiments of this disclosure provide that, where a composition comprises two or more fibers, that each may have a different biodegradation and/or biosorption profile in a physiological fluid, said fluids including water, saline, simulated body fluid, or synovial fluid.

Still other embodiments provide that the polymers, co-polymers, or blends thereof may be photolytically active, such that once electrospun, they may be made to crosslink on exposure to light, thereby improving the tensile characteristics of the scaffold, and increasing the diversity and range of properties available. See for example, Tan, et al., J. Biomed Matl. Res., vol. 87 (4), 2008, pp. 1034-1043, which is incorporated by reference in its entirety.

In some embodiments, the first layer, the second layer, or both, further comprises a porogenic material. At least part of the porogenic material may be present as fibers, particles, or any combination thereof. As used herein, the term “porogen” refers to sacrificial materials added during the production of a scaffold (for example, during electrospinning) and subsequently removed, whose purpose is to occupy space during the construction process, such that their subsequent removal results in what amounts to engineered porosity. In tissue engineering, materials such as inorganic salt like sodium chloride, crystals of saccharose, gelatin spheres or paraffin spheres are used to introduce particulate porosity. In the present disclosure, the use of porogen fibers provides, in some embodiments, porosity aligned with the remaining fibers.

The second population of fibers may include a population of nanoscale bodies disposed within. A nanoscale body may be organic, inorganic (e.g., metallic). A nanoscale body may also be a biological molecule, such as a growth factor, a protease, trypsin, and the like. A variety of dopants may be present within (or on) the fibers of the disclosed materials.

In one set of embodiments, these dopants include at least one therapeutic compound or agent, capable of modifying cellular activity. Similarly, agents that act to increase cell attachment, cell spreading, cell proliferation, cell differentiation and/or cell migration in the scaffold may also be incorporated into the scaffold. Such agents can be biological agents such as an amino acid, peptides, polypeptides, proteins, DNA, RNA, lipids and/or proteoglycans.

These agents may also include growth factors [e.g., a epidermal growth factor, a transforming growth factor-α, a basic fibroblast growth factor, a fibroblast growth factor-acidic, a bone morphogenic protein, a fibroblast growth factor-basic, erythropoietin, thrombopoietin, hepatocyte growth factor, insulin-like growth factor-I, insulin-like growth factor-II, Interferon-β, platelet-derived growth factor, a nerve growth factor, a transforming growth factor, a tumor necrosis factor, Vascular Endothelial Growth Factor, an angiopeptin, or a homolog or combination thereof], cytokines [e.g., M-CSF, IL-1beta, IL-8, beta-thromboglobulin, EMAP-II, G-CSF and IL-IO, or a homolog or combination thereof], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, metalloproteases, ADAMTS 17, tryptase-gamma, and matriptase-2, or a homolog or combination thereof] and protease substrates.

Suitable proteins which can be used along with the present disclosure include, but are not limited to, extracellular matrix proteins [e.g., fibrinogen, collagen, fibronectin, vimentin, microtubule-associated protein ID, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)].

Additionally and/or alternatively, the materials of the present disclosure may comprise an antiproliferative agent (e.g., rapamycin, paclitaxel, tranilast, Atorvastatin and trapidil), an immunosuppressant drug (e.g., sirolimus, tacrolimus and Cyclosporine) and/or a non-thrombogenic or anti-adhesive substance (e.g., tissue plasminogen activator, reteplase, TNK-tPA, glycoprotein IIb/IIIa inhibitors, clopidogrel, aspirin, heparin and low molecular weight heparins such as enoxiparin and dalteparin).

Examples of immunosuppressive agents which can be used to minimize immunosuppression include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNF-α, blockers, a biological agent that targets an inflammatory cytokine, IL-1 receptor antagonists, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

Cytokines useful in the present disclosure include, but are not limited to, cardiotrophin, stromal cell derived factor, macrophage derived chemokine (MDC), melanoma growth stimulatory activity (MGSA), macrophage inflammatory proteins 1 alpha (MOP-1 alpha, 2, 3 alpha, 3 beta, 4, and 5, IL-, 11-2, IL-3, IL-4, IL-5, IL-6, IL-7, IL-8, IL-9, IL-10, IL-11, IL-12, IL-13, TNF-α, and TNF-β. Immunoglobulins useful in the present disclosure include but are not limited to, IgG, IgA, IgM, IgD, IgE, and mixtures thereof. Some preferred growth factors include VEGF (vascular endothelial growth factor), NGFs (nerve growth factors), PFGF-AA, PDGF-BB, PDGF-AB, FGFb, FGFa, and BGF.

It should be understood that the disclosed materials may include a single type of nanoscale body/dopant, or two or more types of nanoscale bodies/dopants.

A nanoscale body suitably has at least one cross-sectional dimension in the range of from about 1 nm to about 100 nm. A cross-sectional dimension is a length, width, diameter, or thickness. A nanoscale body suitably has an aspect ratio in the range of from about 1 to about 100. For example, a nanorod having a height of 10 nm and a diameter of 5 nm would be considered suitable. A nanorod having a diameter in the range of from about 2 to about 4 nm and a length in the range of from about 7 nm to about 9 nm is considered an especially suitable nanoscale body, particularly when the nanorod is made of gold.

One embodiment of the present disclosure provides for the selection of materials for the fibers of sufficiently high modulus such that as one of the fibers degrades, the scaffold retains the required modulus, for example at least 20 MPa, preferably at least 40 MPa, more preferably at least 60 MPa, and more preferably at least about 80 MPa, at a strain region of about 10%, and/or an overall circumferential modulus in the range of about 5 to about 60 MPa, preferably at least 10 MPa, more preferably at least 20 MPa, and most preferably at least about 30-35 MPa, at a strain region of about 3%, as defined herein, for sufficient time, for example over 10-20 weeks, under physiological conditions.

In other embodiments, a first fiber comprises a material characterized as having a yield strain at least about 1%, preferably at least about 4%, more preferably at least about 8% and most preferably at least 10%. This fiber may be biocompatible, but may or may not be biodegradable, though it is preferably so. Absolute tensile properties of this material may beless important than are those of the second fiber, since it is the combination of the moduli of the first and second fibers, especially as a function of time of exposure to physiological conditions, that are important, but to accomplish this, the modulus of the first fiber material should be at least about 20 MPa at lower (3%) strain levels. Higher values are preferred, for example, preferably at least about 100 MPa, and most preferably at least about 200 MPa, especially at higher (10%) strain levels. In one embodiment, the first fiber material comprises poly(caprolactone). In other embodiments, this first fiber comprises a poly(β-amino ester) or an acrylate terminated poly(β-amino ester). Such materials are described, for example, in Anderson, et al., “A Combinatorial Library of Photocrosslinkable and Degradable Materials,” Adv. Materials, vol. 18 (19), 2006, this reference being incorporated by reference in its entirety.

A second fiber may comprise a biodegradable material characterized as having a modulus in the range from about 10 MPa to about 500 MPa, when subjected to a strain in the range of from about 1% to about 10% and measured in the direction of the fiber alignment; in another embodiment, this modulus is in the range of about 20 MPa to about 500 MPa; in another, this modulus is in the range of about 300 MPa to about 500 MPa, especially at higher (10%) strain levels. In another embodiment, the second fiber comprises poly(glycolic acid). In yet another embodiment, the second fiber comprises a blend of poly(caprolactone) and poly(glycolic acid). In other embodiments, this second fiber also comprises a poly(β-amino ester) or an acrylate terminated poly(β-amino ester). Yield stress for this fiber material should be at least 1%, preferably at least about 4%, and most preferably at least about 8%.

One embodiment of the invention provides that the first and second fibers are be biodegradable, and that the rates of biodegradability of the two fibers are different, when subjected to similar or the same physiological conditions. In one embodiment, the second fiber biodegrades more quickly than the first. In this embodiment, when taken together, the relative rates of biodegradability (or biosorption or dissolution) of the first fiber, the second fiber, and the porogen fiber can be considered slow, medium, and fast. It is preferred that the relative lifetime of the second fiber in vivo is sufficiently long so as to provide a sustained basis for tissue regeneration—generally on the order of weeks under physiological conditions. The relative lifetime of the second fiber in vivo can be determined or approximated by measuring tensile properties or weight loss of the scaffold under simulated physiological conditions.

In one embodiment, the scaffolding contains a porogen fiber, co-spun with the first and the second fibers, comprising an amount in the of about 10 to about 80 weight percent based on the total weight of electrospun fibers, preferably in the range of about 20 to about 60 weight percent, more preferably in the range of about 30 to about 60 weight percent, and most preferably in about 40-55 weight percent, all with respect to the total weight of electrospun fibers. As used herein, the term “porogen” refers to sacrificial materials added during the production of a scaffold (for example, during electrospinning) and subsequently removed, whose purpose is to occupy space during the construction process, such that their subsequent removal results in what amounts to engineered porosity. In tissue engineering, materials such as inorganic salt like sodium chloride, crystals of saccharose, gelatin spheres or paraffin spheres are used to introduce particulate porosity. In the present invention, the use of porogen fibers provides porosity aligned with the remaining fibers.

In another embodiment, this porogen fiber has been removed, such that the resulting scaffold contains spacings defined by the absence of this porogen material. This removal can be accomplished in several ways, though the most usual way of doing so is by selective dissolution. Preferably, this porogen fiber is capable of selectively and substantially dissolving in physiological fluids, such as water, saline, meniscal fluid, simulated body fluid, or synovial fluid, such dissolution occurring within one hour, preferably within 30 minutes, and most preferably within 10 minutes of contact with the physiological fluid at ambient or physiological conditions. In such circumstances, one non-limiting example, the porogen fiber comprises poly(ethylene oxide). However, the means of removal is not limited to use of physiological fluids. For example, depending on the porogen, hydrocarbons or other organic solvents may also be used (ex vivo). Also the removal can be accomplished inside or outside the patient. One of the purposes of applying and then removing this porogen fiber material is to provide spaces within the matrix to expedite cellular ingress into the scaffold matrix, whose fiber densities otherwise inhibit this incursion. For this reason, one skilled in the art would appreciate that removing the porogen material from the matrix before seeding with, for example, cell populations, and implanting into the patient can be a desirable scenario.

Other embodiments describe the relative proportion of the first and second fiber. In one such embodiment, the first fiber comprises an amount in the range of about 20% by weight to about 80% by weight, relative to the combined weight of the first and second fiber. Other embodiments define the relative amount of the first fiber to be in the range of about 40% by weight to about 60% by weight, or about 50% by weight, each relative to the combined weight of the first and second fiber. The specific ratio of the two fibers will depend on the particular choice of fibers, and one skilled in the art would be able to understand the most appropriate ratio for a given set of fiber materials based on the teaching herein.

The invention teaches that the electrospun fibers may individually comprise the individual polymers or copolymers, or blends of polymers or copolymers or both. Within the scaffold and/or within the individual fibers, the first fiber material may be present in the range of about 1 to about 80 weight percent, and the second fiber may be present in the range of about 80 to about 1 weight percent, each with respect to the total weight of electrospun fibers.

Together, the first and second fibers may form a scaffold whose circumferential modulus in the range of about 10 MPa to about 100 MPa, preferably in the range of about 60 MPa to about 90 MPa, more preferably in the range of about 70 MPa to about 85 MPa, most preferably about 80 MPa. This is accomplished by combining the fiber materials, applied either as individual fibers or co-spun as blended materials. such that the weighted average of the materials according to their individual moduli provide the target scaffold circumferential modulus. One skilled in the art would be able to measure and/or calculate the combined modulus as a function of such a composite. In the simplest case, this relationship can be characterized according to the Rule of Mixtures equation:

Modulus of the composite=Σ[(φₓ*(modulus of material x)],

where φₓ represents the weight fraction of the x^{th} component (strictly speaking, the rule of mixtures deals with volume fractions, but to a good approximation, and assuming polymers of comparable densities are used, use of weight fractions provides an equivalent means of characterization).

Tensile modulus is a property which is often defined in terms relative to the total cross-sectional area of the fiber or fiber bundle, or in this case, to the circumferential alignment of fibers. So as to maintain internal consistency, as described herein, whether the scaffold contains or has had removed the porogen fiber, the moduli are calculated and described so as to consider the cross-sectional area of the porogen fiber, but not to consider the tensile properties of that porogen fiber. For example, in but one non-limiting example, a mixture comprising 25% by weight (of the total polymer weight) of a first polymer, having a modulus of ca. 20 MPa, and 25% by weight a second fiber, having a modulus of 300 MPa, and 50% by weight of a porogen fiber, having a modulus of 100 MPa is described herein as having a composite modulus of 80 MPa for the composite (i.e., (25%×20 MPa)+(25%×300 MPa)+(50%×0 MPa)=(5+75+0)=80 MPa), and not 130 MPa (as would result if the modulus of the scaffold retained the contribution of the porogen; i.e., (25%×20 MPa)+(25%×300 MPa)+(50%×100 MPa)=(5+75+50)=130 MPa) or 160 MPa (as would result if the cross-sectional area of the porogen were ignored; i.e., (50%×20 MPa)+50%×300 MPa)=(10+150)=160 MPa. It should be appreciated that this definition provides a more rigorous requirement for tensile modulus for the scaffold than if the tensile contribution of the porogen material had been considered or if the cross sectional area of the porogen material had been ignored.

Other embodiments of this invention lift the constraint that the first fiber have a particular yield stress value, and allowing the second fiber to have a modulus lower in value than described above, replacing these requirements with one that the combination of first and second fibers maintain a mean circumferential scaffold modulus of at least about 40 MPa, preferably about 60 MPA, and most preferably at least about 80 MPa, at higher (10%) strain levels, when subjected to physiological fluids under physiological conditions for times sufficient to allow for cell ingress and proliferation, typically on the order of weeks. As described earlier, it is highly desirable that the scaffold maintain a minimum modulus during the time of this cell ingress and proliferation, corresponding to healing.

It is also understood that electrospinning provides fibrous solid bodies which contain a degree of porosity which can be affected by the materials of construction—both fibers and other incorporated materials—and the method of making. Accordingly, certain embodiments of this invention describe porous solids whose void volumes are on the order of about 5 to 99 volume percent; other embodiments describe porous solids with void volumes at the lower end of this range, e.g., in the range from about 5 volume percent to about 25 volume percent; still other embodiments describe porous solids with void volumes in the middle of this range, e.g., in the range from about 25 volume percent to about 75 volume percent; and still other embodiments describe solids with void volumes at the high end of this range, e.g., in the range from about 50 volume percent to about 95 volume percent. Other exemplary sub-ranges contemplated by the present invention include the range of about 80 to about 99 volume percent, the range of about 85 to about 95 volume percent, and the range of about 90 to about 95 volume percent.

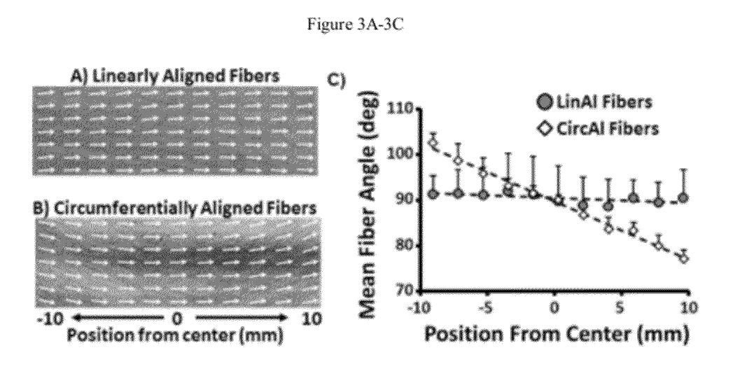

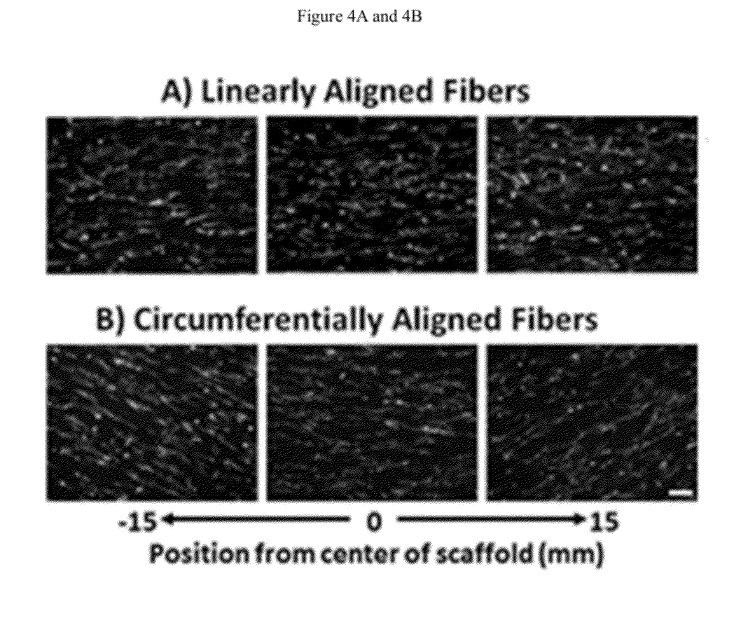

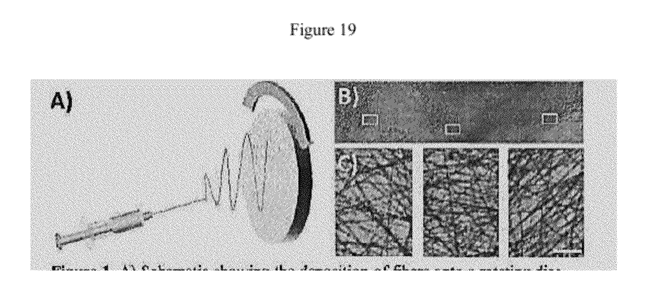

In the disclosed compositions, at least a portion of the first population of fibers are suitably substantially aligned in a first direction. It should be understood that not all of the first population of fibers need be aligned in this first direction, which first direction may be characterized as being about circumferential to a hypothetical central axis, as illustrated in, e.g., FIG. 3b, FIG. 4b, and FIG. 19. It is preferable that more than 50% of the fibers be aligned in this first direction, but 50% should not be understood as being a particular threshold. Similarly, at least a portion of the second population of fibers are substantially aligned in a second direction. The first a second directions may be parallel to one another, although parallel directions are not a requirement. In some embodiments, at least a portion of the second population of fibers is aligned perpendicular to at least a portion of the first population of fibers. In this way, the perpendicular fibers may act as tie fibers in an artificial meniscus material.

In some preferred embodiments, at least a portion of the first population of fibers, at least a portion of the second population of fiber, or both, have an arcuate alignment. Arcuate should be understood as referring to fibers that are curved (as opposed to being straight). The curve may be a circular or circumferential one, although other curves (e.g., elliptical or other curves that are not based on a constant radius) are also suitable.

Illustrative fibers are shown in FIG. 3, which shows linearly aligned fibers (FIG. 3A) and circumferentially-aligned fibers (FIG. 3B). FIG. 3C illustrates the mean fiber angle (degrees) as a function of the fiber's position from the center of the mandrel. Data are shown for linearly-aligned fibers (which have an unchanging fiber angle) and for circularly-aligned fibers (which have an angle that changes as a function of the fiber's distance from the center of the mandrel).

The first population of fibers, the second population of fibers, or both, may have an anisotropic alignment. In some embodiments, the compositions have a fiber alignment that varies spatially within the body of the composition. This is described below in additional detail.

At least some of the first population of fibers may differ from at least some of the second population of fibers in composition, cross-sectional dimension, or both. For example, some of the first population of fibers may have an average diameter of about 100 nm, and some of the second population of fibers may have an average diameter of about 150 nm. The first population may be formed from a first biocompatible polymer, and the second population may be formed from a second, different biocompatible polymer. By changing the composition, cross-sectional shapes, or dimensions of the fibers throughout the spinning process, it is possible to provide solids wherein the different layers are comprised of different compositions, cross-sectional shapes, or thicknesses. Several means to achieve this alignment are described in Baker and Mauck, “The effect of nanofiber alignment on the maturation of engineered meniscus constructs,” Biomaterials, 28 (2007) 1967-1977, which is incorporated in its entirety by reference for this purpose.

In some embodiments, at least one of the first layer and the second layer has a population of cells disposed thereon. These populations of cells can exist within the composition as homogeneous or heterogeneous mixtures, and as at least one gradient either across a radial and axial/longitudinal distance, or both. These gradients can be continuous or step-wise, as with the other components, as determined by the processing parameters.

Techniques for seeding cells onto or into a scaffold are well known in the art, and include, without being limited to, static seeding, filtration seeding and centrifugation seeding. See, e.g., Baker and Mauck, “The effect of nanofiber alignment on the maturation of engineered meniscus constructs,” Biomaterials, 28 (2007) 1967-1977, which is incorporated in its entirety by reference for this purpose. Static seeding includes incubation of a cell-medium suspension in the presence of the scaffold under static conditions and results in non-uniform cell distribution (depending on the volume of the cell suspension); filtration seeding results in a more uniform cell distribution; and centrifugation seeding is an efficient and brief seeding method (see for example, EP19980203774).

The cells may be seeded directly onto the scaffold, or alternatively, the cells may be mixed with a gel which is then absorbed onto the interior and exterior surfaces of the scaffold and which may fill some of the pores of the scaffold. Capillary forces will retain the gel on the scaffold before hardening, or the gel may be allowed to harden on the scaffold to become more self-supporting. Alternatively, the cells may be combined with a cell support substrate in the form of a gel optionally including extracellular matrix components.

The cells which can be used according to the teachings of the present disclosure may comprise non-autologous cells or non-autologous cells (e.g. allogeneic cells or xenogeneic cells), such as from human cadavers, human donors or xenogeneic (e.g. porcine) donors.

The cells may comprise a heterogeneous population of cells or alternatively the cells may comprise a homogeneous population of cells. Such cells can be, for example, stem cells (such as adipose derived stem cells, embryonic stem cells, bone marrow stem cells, cord blood cells, mesenchymal stem cells, adult tissue stem cells, induced pluripotential stem cells,), progenitor cells (e.g. progenitor bone cells), or differentiated cellarrays such as chondrocytes, meniscal fibrochondrocytes, osteoblasts, osteoclasts, osteocytes, connective tissue cells (e.g., fibrocytes, fibroblasts, tenocytes, and adipose cells), endothelial and epithelial cells, or mixtures thereof.

As used herein, the phrase “stem cell” refers to cells which are capable of differentiating into other cell types having a particular, specialized function (i.e., “fully differentiated” cells) or remaining in an undifferentiated state hereinafter “pluripotent stem cells”.

Furthermore, such cells may be of autologous origin or non-autologous origin, such as postpartum-derived cells (as described in U.S. application Ser. Nos. 10/887,012 and 10/887,446). Cells may be selected according to the tissue being generated.

It will be apparent that the disclosure has applications beyond medical tissue engineering. For example, certain embodiments comprise industrial applications, including dimensionally shaped filters, textile, composites, catalyst scaffolds, electrolytic cell diaphragms, battery separators, and fuel cell components.

Certain other embodiments provide that the fibrous material also comprises a catalyst, including biological catalysts, for example, but not limited to, enzymes, anti-microbials, or anti-fungals. The means for incorporating these catalysts onto or within electrospun materials are well known in the art as being analogous to those used to incorporate particles as described above. The means for functionalizing, immobilizing and/or attaching catalysts onto polymer materials are equally known to those skilled in the art. The first and second layers may have different populations of cells disposed thereon. As an example, the first layer may include stem cells, and the second layer may include connective tissue cells.

The present disclosure also provides methods. These methods include irradiating a first fibrous layer comprising a first population of polymeric fibers having a first population of nanoscale bodies disposed within, the irradiating being performed so as to bond at least a portion of the first layer to a second fibrous layer comprising a second population of polymeric fibers. Without being bound to any single theory, one may consider the irradiating as causing local heating at or around the nanoscale bodies so as to bond the first and second fibrous layer together at or near to the site of the irradiation. It should be understood that the first and second layers may both contain populations of nanoscale bodies.

Suitable nanoscale bodies are described elsewhere herein. As explained above, a nanoscale body suitably has at least one cross-sectional dimension in the range of from about 1 nm to about 100 nm, or even in the range of from about 10 nm to about 50 nm.

The irradiating may be effected by lasing, suitably by an infrared laser. An exemplary laser is described elsewhere herein. The user of ordinary skill in the art will be equipped to select a laser of a suitable wavelength to effect the desired local heating.

In some embodiments, a user may dispose the first population of nanoscale bodies within a polymeric fluid so as to form a first mixture and then electrospin the first population of polymeric fibers from the first mixture. Additional information concerning electronspinning is presented elsewhere herein. A user may further dispose the second population of nanoscale bodies within a polymeric fluid so as to form a second mixture and then form the second population of polymeric fibers (e.g., via electrospinning) from the second mixture.

The populations of polymeric fibers may be, as described elsewhere herein, formed from one or more material that is natural, synthetic, biocompatible, biodegradable, non-biodegradable, biosorbable, or some combination thereof.

The first population of fibers, the second population of fibers, or both, has an average cross-sectional dimension in the range of from about 10 nm to about 10,000 nm, or in the range of from about 100 nm to about 1000 nm, or from about 200 nm to about 500 nm.

The first population of polymeric fibers, the second population of polymeric fibers, or both, may be characterized as being an anisotropic alignment of fibers. The fiber alignment may be linear, but may also be arcuate, as described elsewhere herein. In some embodiments, at least some of the fibers in the first layer have a different alignment than some of the fibers of the second layer. In some embodiments, at least some of the fibers in the second layer are oriented essentially perpendicular to at least some of the fibers of the first layer.

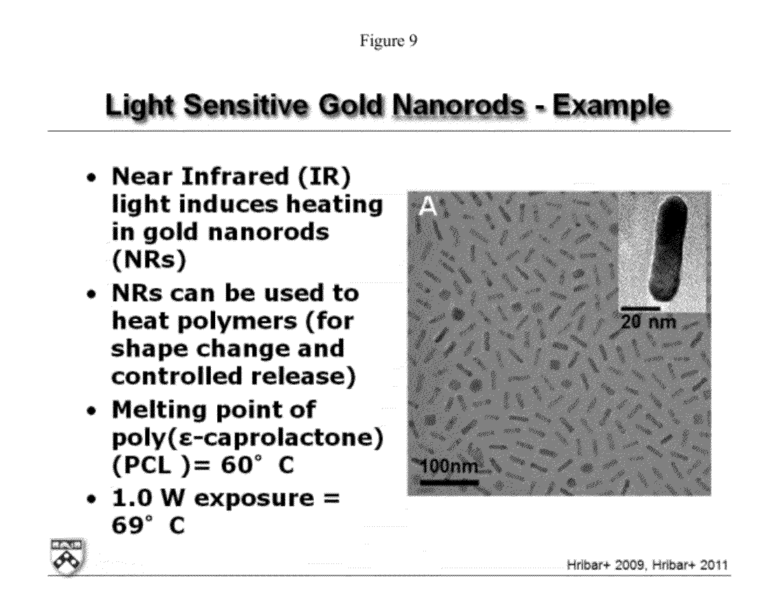

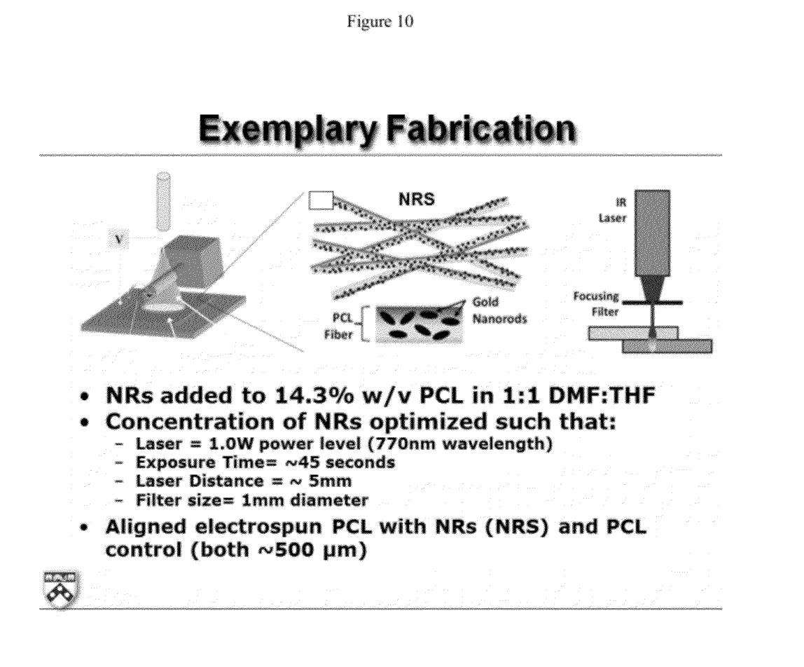

FIG. 9 presents exemplary process conditions for a fibrous layer that contains nanoscale bodies. As shown in the figure, nanoscale gold nanorods may be disposed within a polymer. Application of radiation (e.g., laser) may then effect local heating so as to raise the temperature of the polymer at the site of the irradiation to around or above the melting temperature of the polymer, thus allowing the user to melt together adjacent layers of material. Further information is provided in FIG. 10, which figure presents an exemplary scheme for forming a laminate. As shown in the figure, gold nanorods (NRs) were added to poly-caprolactone (PCL) polymer, which polymer was then electrospun into layers. The layers were then irradiated with a laser at 1 W of power at 770 nm wavelength for about 45 seconds at a distance of about 5 mm with a focusing filter in place. The irradiation resulted in local fusion between adjacent layers.

Also provided are methods. These methods include electrospinning, from a polymeric fluid, a first population of polymeric fibers onto a first rotating surface of a mandrel, the electrospinning being performed such that at least a portion of the first population of polymeric fibers is aligned on the first surface in an arcuate fashion.

In some embodiments, a spinneret containing the polymeric fluid is oriented essentially perpendicular to the plane of the first rotating surface of the mandrel. The spinneret may, in some embodiments, be oriented essentially parallel to an axis about which the first rotating surface of the mandrel rotates. The spinneret need not be oriented exactly parallel to the mandrel's axis of rotation, and the spinnarent need not be aligned so that it is in register with the axis of rotation. For example, in the case of a disc-shaped mandrel, the spinneret may be aligned with a point that is at a radial distance from the center of the disc. The spinneret may, of course, be aligned with the center of the disc.

In some preferred embodiments, such as the embodiment shown in FIG. 19A, the spinneret dispenses polymer so as to electrospin a fiber onto a rotating mandrel. The mandrel suitably has an angular velocity, which in turn allows the fibers to align in a circumferential direction. As explained elsewhere herein, fibrous materials (which may be termed “scaffolds”) formed according to these methods may have a spatially varying macrostructure. The mandrel is suitably circular, although circular mandrels are not necessary. The surface of the mandrel also need not be perpendicular to the spinneret. Fibrous materials may be formed in an arrayed fashion, in which multiple mandrels rotate as multiple spinnarets dispense polymer onto the mandrels so as to simultaneously form fibrous layers on the mandrels.

The polymeric fibers may be formed of a polymer that is natural, synthetic, biocompatible, biodegradable, non-biodegradable, biosorbable, or any combination thereof, as described elsewhere herein. Fibers that comprise polycaprolactone are considered especially suitable.

A user may also deposit a cell or even a population of cells onto the electrospun fiber. It should be understood that this deposition is not limited to applying the cells to a surface of the fiber, as a user may contact or otherwise immerse the fiber into a cell-containing medium. Stem cells and other cells (including those cells described elsewhere herein) are all considered suitable for this application.

At least a portion of the mandrel surface onto which the fiber is electrospun suitably has a linear velocity during electrospinning of between about 8 m/s and about 12 m/s; linear velocities of about 10 m/s are considered especially suitable.

The polymeric fiber may be electrospun so as to form a body having at least one cross-sectional dimension in the range of from about 10 micrometers to about 1 cm, or from about 50 micrometers to about 0.5 cm. Bodies formed according to these disclosed methods are suitably fibrous layers in form. The fibrous layers may then be cut to conform to a shape that a user desires.

In some embodiments, the rotating surface of the mandrel comprises a first conductive region and a second conductive region separated by an insulating region disposed there between. Such a pattern may be characterized as being a target or bulls-eye pattern. These patters may be used to give rise to a plurality of polymeric fibers that are aligned radially relative to an axis about which the first rotating surface of the mandrel rotates. These radially aligned fibers may then be disposed adjacent to fibers that have a arcuate (e.g, circumferential) alignment, with the radially aligned fibers acting as “tie fibers” relative to the arcuately aligned fibers.

The present disclosure also provides compositions. These compositions suitably include a first layer comprising a first population of polymeric fibers, the first population of polymeric fibers having an anisotropic alignment that varies spatially within the layer. An exemplary material is shown in FIG. 19B and FIG. 19C, which figures show spatial variation of fiber alignment within a fibrous material. At least a portion of the first population of polymeric fibers has an arcuate alignment; fibers having a circular or circumferential alignment are considered especially suitable. At least a portion of the first population of fibers suitably has an average diameter in the range of from 10 nm to about 10 micrometers. The population of fibers may contain a population of nanoscale bodies; suitable such bodies are described elsewhere herein.

The disclosed compositions may also include a population of cells contacting the first population of fibers. The cells may be stem cells, connective tissue cells, or other cells described elsewhere herein. The polymeric fibers may be formed from materials that are natural, synthetic, biocompatible, biodegradable, non-biodegradable, and/or biosorbable, and unless specifically restricted to one or more of these categories, the fibers may comprise materials from any one of these categories.

Compositions according to this disclosure may include a second fibrous layer, the second fibrous layer comprising a second population of fibers. The second fibrous layer is suitably bonded to the first fibrous layer. The second fibrous layer may comprise a second population of fibers having an average diameter in the range of from 10 nm to about 10 micrometers. The second population of fibers may be aligned essentially parallel to one another. In some embodiments, the second population of fibers has an arcuate alignment. The alignments of the first and second populations of fibers may be different; i.e., the second population of fibers is aligned in a direction that differs from the alignment of the first population of fibers. The second population of fibers may include a population of nanoscale bodies disposed within. Suitable such bodies are described elsewhere herein. The compositions may include a porogenic material. The porogenic material may be disposed within the first layer, the second layer, or even both.

The porogenic materials may be used, as one example, to give rise to a material that includes anisotropically aligned fibers and also includes sufficient void space so allow for cell seeding or even cell ingrowth. As one example, a use may form a fibrous layer from two electrospun polymer materials—a first material that is comparatively fast to degrade under certain conditions, and a second material that is slower to degrade under conditions that degrade the first material. The user may form the desired layer from these two materials; by dispensing both materials from the same spinneret (or even from different spinnarets) and applying the methods disclosed herein, the user can create a fibrous layer that includes fibers of both materials, the fibers all sharing the same alignment. The user may then expose the fibrous material to conditions that degrade the first material, the degradation of that first material in turn giving rise to void spaces within the fibrous layer. Cells may then be seeded into or grow into the void spaces left behind by the now-degraded first material.

The user may then, depending on their needs, install the seeded implant into a subject. The second material of the implant may be chosen to degrade over time under physiological conditions so as to allow the subject's own cells to take the place of the second material as that material degrades, while still providing rigidity to the implant before degradation. Alternatively, the second material may be one that does not degrade under physiological conditions.

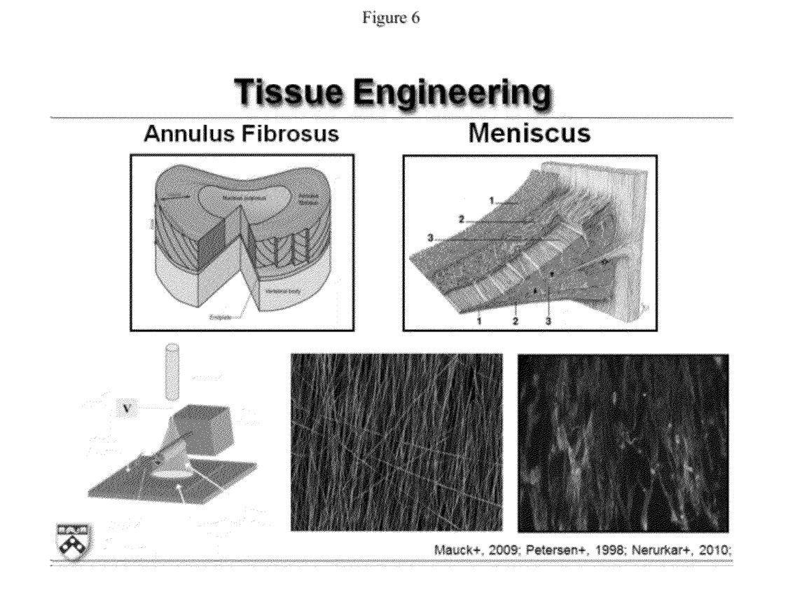

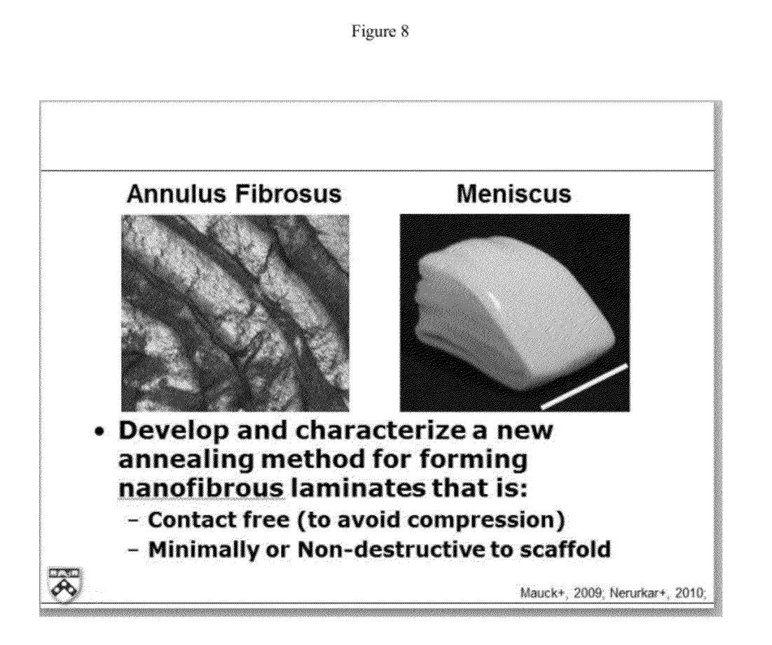

The present disclosure also provides biocompatible implants. These implants suitably include a quantity of a composition described herein, e.g., a composition that has a first layer comprising a first population of polymeric fibers, the first population of polymeric fibers having an anisotropic alignment that varies spatially within the layer. The quantity of material may be shaped so as to approximate at least a portion of a knee meniscus, at least a portion of an annulus fibrosis, or other fibrous tissue. Examplary annulus fibrosis and meniscus tissues are shown in FIG. 6 and FIG. 8, which figures illustrate the fibrous, laminate structure within the tissues.

The present disclosure also provides methods, which method include seeding a composition described herein with a population of cells. The cells may be autologous, non-autologous, or both. The user may then implant the seeded composition into a subject.

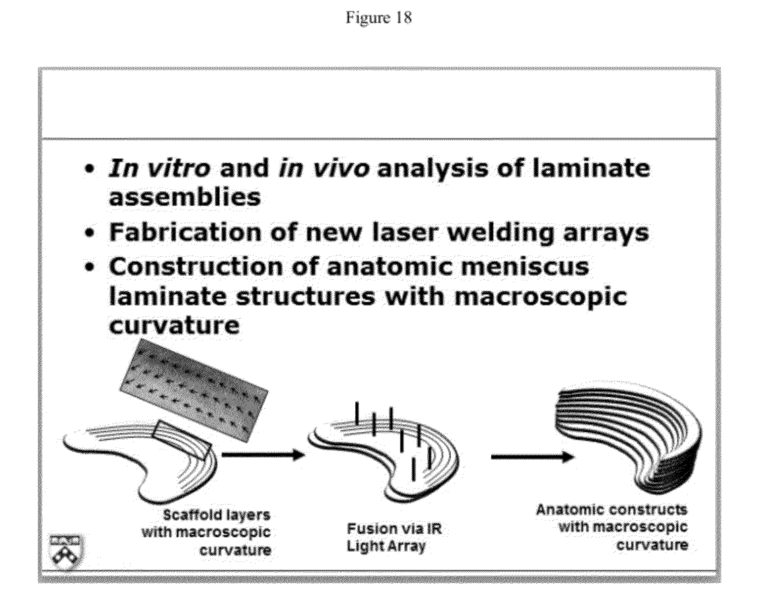

Non-limiting FIG. 18 is illustrative of these methods. As shown in the figure, a user may form one or more fibrous layers, which layers include fibers having an anisotropic alignment, and which fibers may also include nanoscale bodies. The user may then cut the layers to shape. As shown in FIG. 18, the layers may be cut into crescent shapes and then fused to one another by application of laser radiation to the layers, which radiation in turn effects local heating and bonds the layers together. The application of radiation may be performed in an arrayed fashion, where radiation is simultaneously applied to the fibrous layers at a variety of locations, e.g., by an array of lasers. Alternatively, radiation may be applied sequentially to a variety of locations.

By cutting the layers to different shapes and laying the layers atop one another, the user may then “build up” a three-dimensional body that has an anatomic form and that also features arcuately aligned fibers within, the alignment of the fibers replicating the alignment of the fibers in naturally-occurring tissue. The user may seed the layers with cells before, during, or even after the layers are bonded to one another so as to form the anatomic construct.

### EXEMPLARY EMBODIMENTS

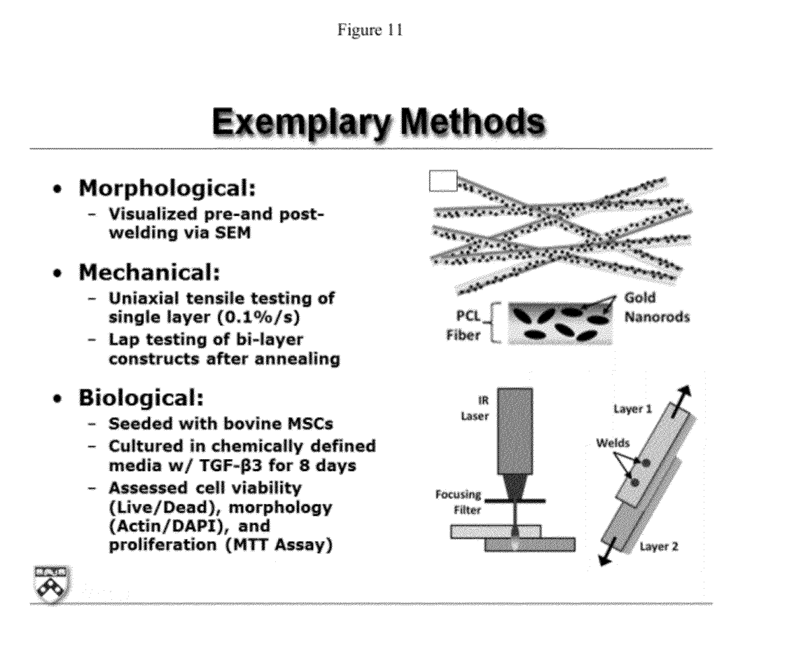

The following results are illustrative only and do not limit the scope of the disclosure. Exemplary processing and testing schemes are outlined in FIG. 10 and FIG. 11.

Thermal Bonding

Gold nanorods (NR) were fabricated. NR were suspended in a solution of poly(ε-caprolactone) (PCL) in 1:1 THF:DMF at varying concentrations prior to electrospinning into thin (˜500 μm thick) fibrous scaffolds. Optimization of NR concentration was carried out on 10 mm×10 mm strips using a handheld IR laser (Chameleon Ti: sapphire laser, 770 nms, 1.0 W) distanced ˜5 mm from the sample. IR light was focused through a ˜1 mm diameter filter, and exposure time was set at ˜45 seconds. Nanorod-containing scaffolds (NRS) and PCL-alone scaffolds (PCL) were sputter coated and viewed under SEM. Fiber size was determined using ImageJ. For mechanical testing, scaffolds were extended to failure at 0.1%/sec using an Instron 5848. Laminates were formed by overlaying 20×10 mm scaffold strips, with a 10 mm overlap region. Fusion of layers was accomplished (with one or two weld points) via a heated probe or via IR exposure as above. Laminates were extended to failure at a rate of 0.1%/sec, and maximum load of the interface recorded. Bovine MSC viability and morphology was assessed on NRS with and without prior IR exposure via Live/Dead and Actin-DAPI staining through day 7. Statistical significance was determined by ANOVA with Tukey's post-hoc tests (p<0.05).

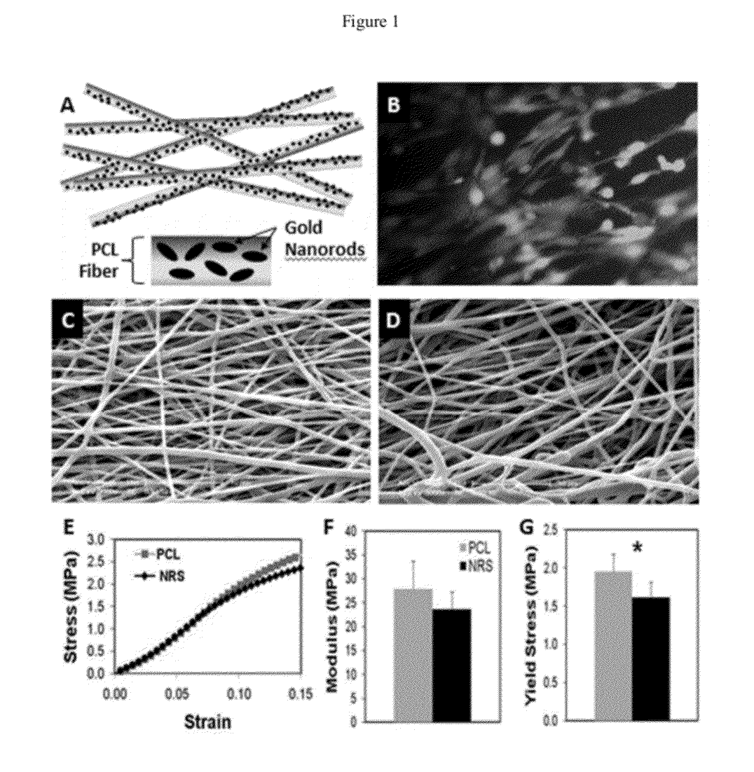

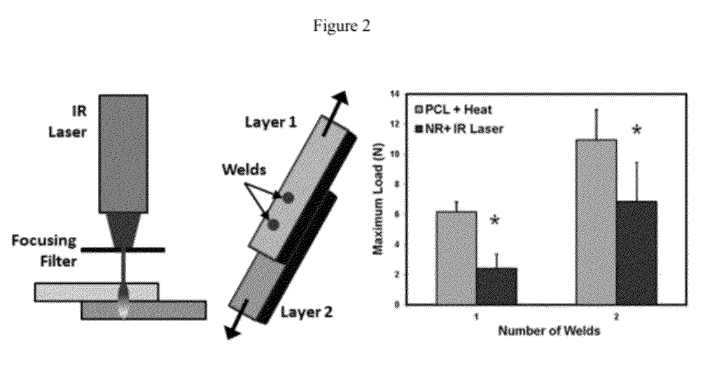

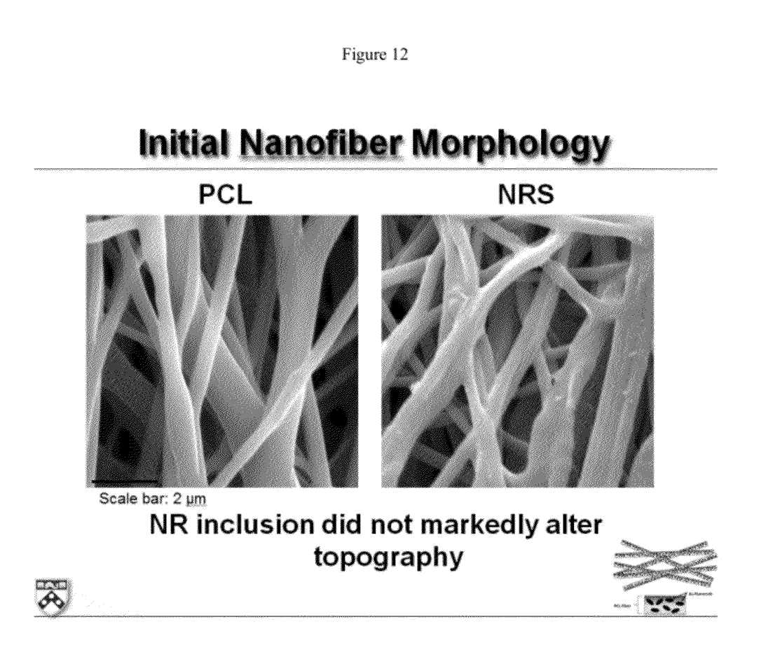

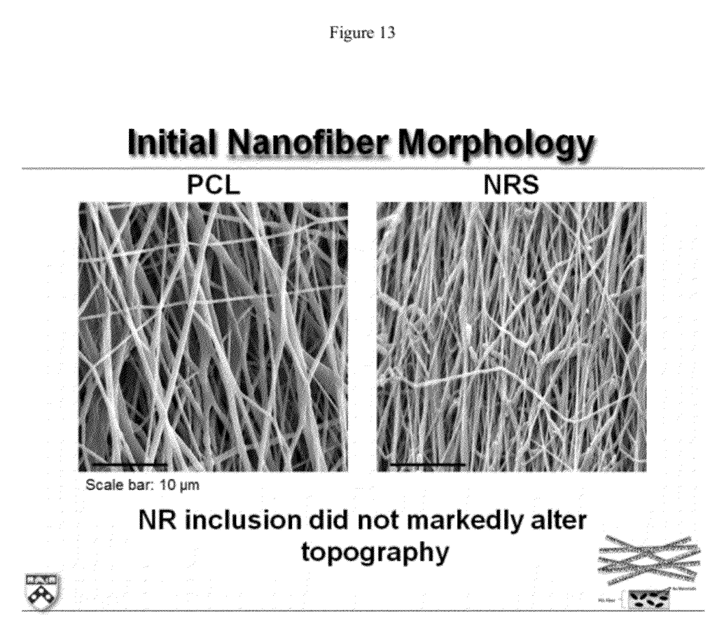

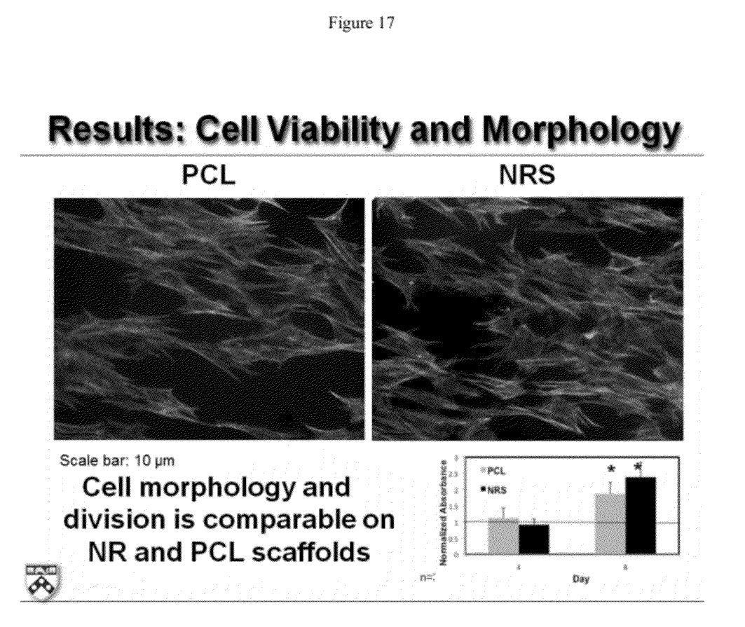

Inclusion of gold nanorods within PCL fibers (FIG. 1A) had no effect on fiber morphology, as shown in FIG. 1C and FIG. 1D, as well as by FIGS. 12, 13, and 17. Cell viability and shape on the NRS appeared normal, with cells elongated in the fiber direction (FIG. 1B). Further, NRS modulus did not differ from PCL (FIG. 1F), though a decrease in yield stress was observed (p<0.05) (FIG. 2G). Heat generation was essentially proportional to NR concentration; the minimum NR concentration required to melt fibers locally was 6.3×10⁻¹⁴ mol NR/mL.

Laminates were readily formed with IR exposure of NRS, with both one and two weld points (FIG. 2). For both annealing methods, increasing weld number increased interface strength (FIG. 2), with spot welding via heated probe having ˜2-3-fold higher strength than laser-mediated assembly (p<0.05). Outside of the welds, cell number and morphology were similar for both methods (not shown). It should be understood that a user may irradiate adjacent layers at one, two, or more locations.

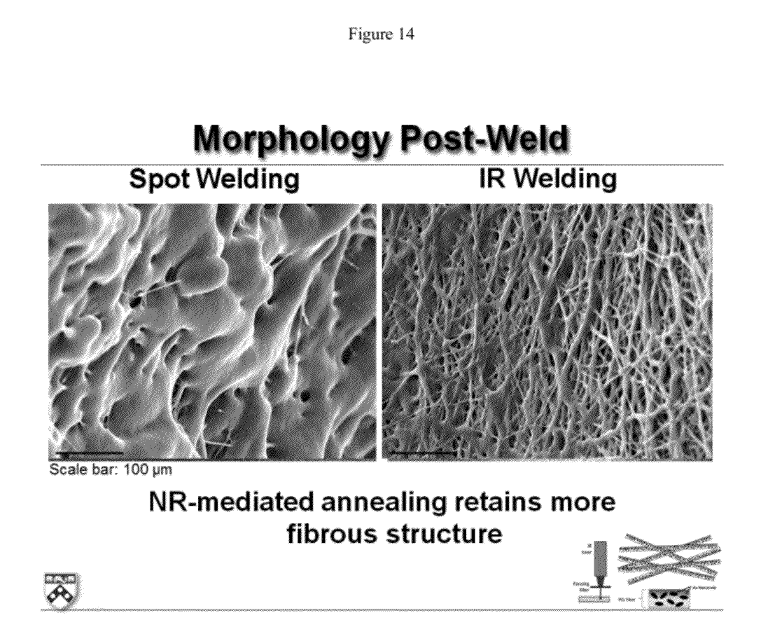

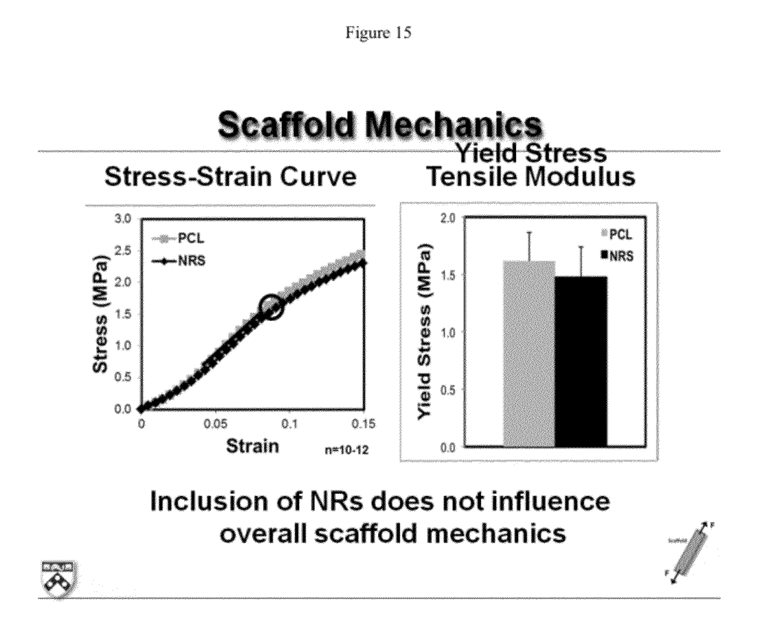

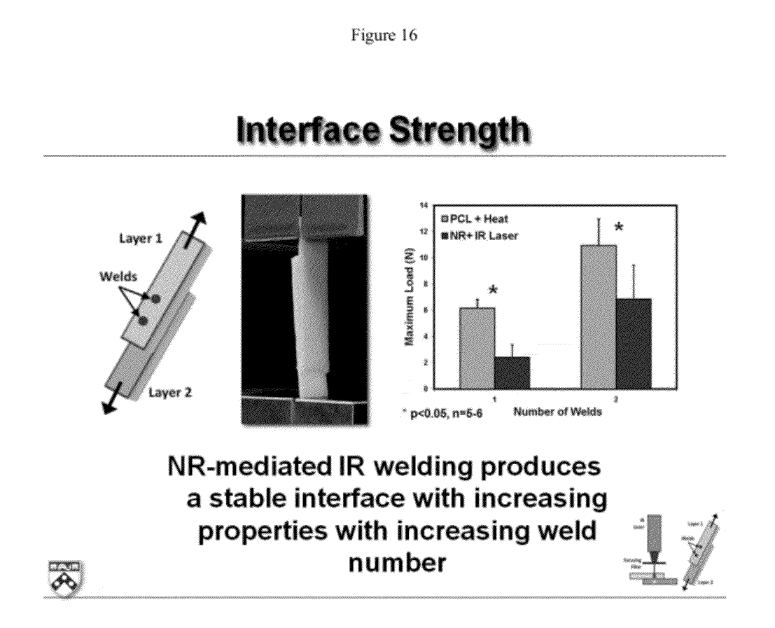

NRs incorporated into electrospun PCL fibers did not substantially alter fiber morphology or scaffold mechanical properties, as shown in FIG. 15. As shown by FIG. 14, NR-irradiation welding retained more fibrous structure than did welding layers using a heated probe. Local heating of gold NRs could be achieved with focused application of IR light and by varying the concentration of NRs within the spinning solution. Inclusion of NRs within the fibers did not appear to alter cell viability or morphology after one week of culture. Consistent with the previous annealing method (heated probe), cell morphology was only perturbed within the weld area, suggesting normal fiber architecture outside the exposure site. (see FIG. 14) Welds were capable of holding layers in apposition for a long enough period of time for cell colonization and matrix deposition to occur. As shown in FIG. 16, nanorod-mediated infrared welding produced a stable interface, which interface had increasing mechanical properties with increasing weld number.

Moreover, as the NR-mediated method does not require physical contact with the scaffold, local compression is not a concern. This process is applicable to the assembly of anatomic laminate structures for engineering complex tissues such as the knee meniscus and annulus fibrosis.

Electrospun Scaffolds

Scaffold Fabrication:

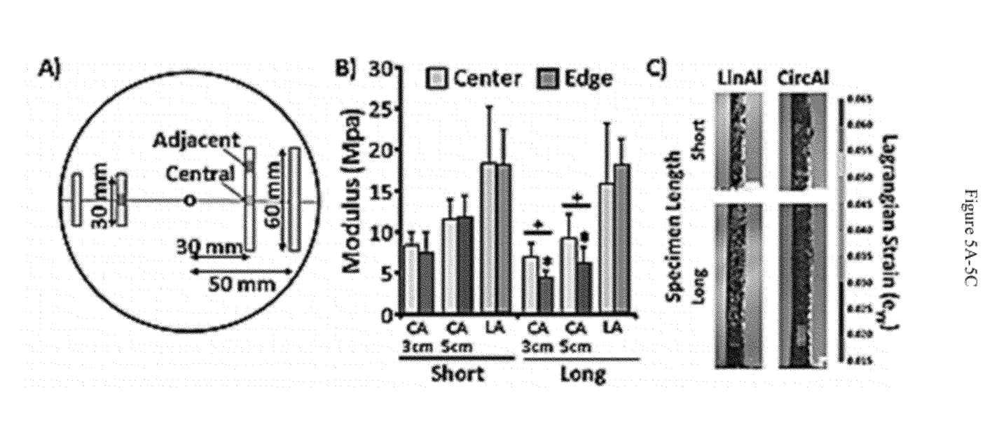

Electrospinning of poly(-caprolactone) (PCL) nanofibers was carried out to form linearly aligned fiber scaffolds. Additionally, circularly aligned (CircAl) PCL scaffolds were formed by modifying the collection apparatus such that the polymer was centered above and perpendicular to the plane of a grounded rotating aluminum collecting plate (FIG. 5A).

Fiber Alignment:

To quantify the alignment of fibers, thin films were collected for ˜5 minutes on glass slides (n=6/scaffold type) and viewed by light microcopy (Eclipse 90i, Nikon Instruments). For CircAl fibers, fibers were collected at a radius of 3 cm on the mandrel. Images were collected at 4× magnification over a 20 mm by 10 mm area using a motorized stage and an image stitching program (NIS Elements software, version 3.22). Fiber alignment of individual images was quantified via a custom MATLAB script utilizing Fast Fourier Transform analysis. Mean fiber alignment was determined as a function of spatial position on the slide (FIG. 3A). For statistical analysis, Pearson correlation coefficients were calculated between mean fiber alignment and spatial location, with significance set at p<0.05. FIG. 3B illustrates the circumferential alignment of fibers as a function of the fibers' distance from the center of the rotating mandrel on which the fibrous body was formed.

Cellular Interactions:

Additional scaffolds (30 mm×10 mm, n=3) were utilized to assess cellular morphology. Juvenile bovine mesenchymal stem cells were harvested and cultured up to P2 as previously described (5). Cells (80,000) were deposited on each scaffold, cultured for 5 days, and fixed in 4% paraformaldehyde. Cell nuclei and F-actin were visualized with 4′,6-diamidino-2-phenylindole (DAPI) and phalloidin-Alexa488 (Invitrogen), respectively, and imaged on a fluorescent microscope (Eclipse 90i, Nikon Instruments).

Mechanical Testing:

Oriented scaffolds of ˜0.7-0.8 mm thickness were formed (n=7/scaffold type). For the CircAl scaffolds, specimens were excised from oriented mats at two radial positions (3 cm and 5 cm). All scaffolds were excised at two lengths (30 and 60 mm) with a 3 mm width (FIG. 5A, n=7/group). The cross-sectional area of the samples was calculated using a custom laser-based device. Samples were then sputter coated with Verhoff's stain and placed in custom-made grips. Clamp-to-clamp length was set at 15 mm and 45 mm for the short and long strips, respectively. Mechanical evaluation was performed using a materials testing machine (Instron, model 5848) in a saline bath at 37° C. Samples were preloaded to 0.5N, subjected to 15 cycles of preconditioning from 0-3%, and then extended to failure at a rate of 0.5%/sec. Images were acquired at a rate of 2 frames per second. Lagrangian strain in an area near the scaffold center and an area of equal size near the grips were computed using Vic2D (Correlated Solutions). Tensile modulus for each region was calculated as the slope of the stress-strain curve between 1-3% strain. For statistical analysis within each scaffold type, a two-way repeated measures ANOVA was performed, followed by paired t-tests to determine statistical differences between individual groups. Overall statistical significance was maintained at p<0.05.

By collecting fibers on a rapidly revolving surface, organized nanofibrous scaffolds with pronounced curvature could be formed (FIG. 3). Fiber orientation varied linearly across a 20 mm distance, ranging from ˜103° to ˜77° at a radius of 3 cm. Statistical analysis confirmed this relationship with a Pearson correlation coefficient of −0.963 (p<0.05). Alternatively, linearly aligned (LinAl) fibers were centered at 90° and did not vary with position (R=−0.111, p>0.05).

When cells were seeded on CircAl and LinAl scaffolds, cytoskeletal and nuclear morphology was similar (FIG. 4). The cells, however, followed the local directionality of the scaffold, with alterations in cellular alignment on a macroscopic scale clearly observed on the CircAl scaffolds (FIG. 4B). As seen in FIG. 3A, the cellular alignment was essentially unchanged along the length of a linearly-aligned fiber

Mechanical analysis of the CircAl scaffolds revealed significant interactions between scaffold length and region within the scaffold (p<0.05, FIG. 5B). As such, comparisons between groups were done on an individual basis. For short specimens, no significant differences were found in the modulus between the center and edge regions of the CircAl specimens (p>0.01). Conversely, for long specimens, the modulus was 32-36% lower near the edge of the scaffolds compared to the center (p<0.01). In terms of scaffold length, the modulus of the central region of the CircAl specimens was similar between short and long specimens (p>0.01); however, the modulus of the edge region for the short CircAl specimens was 68-90% higher than the respective long specimens (p<0.01). No statistically significant differences could be detected between LinAl specimens as a result of varying specimen length or region within the scaffolds (p>0.05). These differences in modulus are further highlighted by a graphical depiction of the Lagrangian strain along the direction of loading (FIG. 5C).

Thus, the disclosed methods allow for the construction of electrospun nanofibrous scaffolds with a spatially varying macroscopic fiber orientation. This orientation is similar to the meniscus. On the microscopic scale, these fibers were locally aligned, allowing alignment of MSCs (mesenchymal stem cells) similar to linearly aligned scaffolds. The CircAl fibers followed a pattern roughly parallel to the circumferential direction of the disc, creating a linear gradient in fiber angle along the length of excised scaffolds and resulting in MSCs with a similar change in morphology over a macroscopic scale, in support of our hypothesis.

The CircAl fiber scaffolds behaved similarly to linearly aligned scaffolds over a short spatial domain, but varied considerably as specimen length increased. Without being bound to any single theory, increasing the length of specimens for tensile testing may increase the frequency of CircAl fibers that enter and/or exit before reaching the clamped regions, resulting in greater regional differences within the scaffolds (i.e. decrease in modulus near the edge). These layers may be combined as shown in FIG. 18 to generate anatomic 3D implants that recreate the macroscopic and microscopic features of the native tissue. The organized nanofibrous scaffolds generated herein replicate the macroscopic curvature of the native tissue, and can direct the formation of an anatomic construct with direction dependent mechanical properties that vary across a large anatomic expanse.

What is claimed:

1. A composition, comprising:
a first layer comprising a first population of polymeric fibers,
at least some of the first population of polymeric fibers comprising nanoscale bodies disposed within; and
a second layer comprising a second population of polymeric fibers,
the first and second layers being bonded together at one or more locations.

2. The composition of claim 1, wherein the first population of polymeric fibers comprises a polymer that is natural, synthetic, biocompatible, biodegradable, non-biodegradable, bioabsorbable, or any combination thereof.

3. The composition of claim 1, wherein the first layer, the second layer, or both, further comprises a porogenic material.

4. The composition of claim 1, wherein at least some of the second population of polymeric fibers comprise nanoscale bodies disposed within.

5. The composition of claim 1, wherein a nanoscale body comprises at least one cross-sectional dimension in the range of from about 1 nm to about 100 nm.

6. The composition of claim 5, wherein a nanoscale body comprises a body having an aspect ratio in the range of from about 1 to about 100.

7. The composition of claim 5, wherein a nanoscale body comprises an organic material, an inorganic material, or both.

8. The composition of claim 7, wherein the inorganic material comprises a metal.

9. The composition of claim 8, wherein the metal comprises gold.

10. The composition of claim 1, wherein at least a portion of the first population of fibers are substantially aligned in a first direction.

11. The composition of claim 10, wherein at least a portion of the second population of fibers are substantially aligned in a second direction.

12. The composition of claim 11, wherein the first and second directions are non-parallel relative to one another.

13. The composition of claim 12, wherein at least a portion of the second population of fibers is aligned perpendicular to at least a portion of the first population of fibers.

14. The composition of claim 1, wherein at least a portion of the first population of fibers, at least a portion of the second population of fiber, or both, have an arcuate alignment.

15. The composition of claim 1, wherein the first population of fibers, the second population of fibers, or both, has an anisotropic alignment.

16. The composition of claim 1, wherein the at least some of the first population of fibers differ from at least some of the second population of fibers in composition, cross-sectional dimension, or both.

17. The composition of claim 1, wherein at least one of the first layer and the second layer has a population of cells disposed thereon.

18. The composition of claim 17, wherein the first and second layers have different populations of cells disposed thereon.

19. The composition of claim 1, wherein the first population of fibers, the second population of fibers, or both, has an average cross-sectional dimension in the range of from about 10 nm to about 10,000 nm.

20. The composition of claim 1, wherein bonding between the layers is effected by mediated matrix-deposition with appositional culture.

21. A method, comprising:
irradiating a first fibrous layer comprising a first population of polymeric fibers having a first population of nanoscale bodies disposed within, the irradiating being performed so as to bond at least a portion of the first layer to a second fibrous layer comprising a second population of polymeric fibers.

22. The method of claim 21, wherein the second fibrous layer comprises a second population of nanoscale bodies disposed within.

23. The method of claim 21, wherein a nanoscale body has at least one cross-sectional dimension in the range of from about 1 nm to about 100 nm.

24. The method of claim 21, further comprising disposing the first population of nanoscale bodies within a polymeric fluid so as to form a first mixture and electrospinning the first population of polymeric fibers from the first mixture.

25. The method of claim 22, further comprising disposing the second population of nanoscale bodies within a polymeric fluid so as to form a second mixture and electrospinning the second population of polymeric fibers from the second mixture.

26. The method of claim 21, wherein the first population of polymeric fibers, the second population of fibers, or both, comprises a material that is natural, synthetic, biocompatible, biodegradable, non-biodegradable, biosorbable, or any combination thereof.

27. The method of claim 21, wherein the first population of fibers, the second population of fibers, or both, has an average cross-sectional dimension in the range of from about 10 nm to about 10,000 nm.

28. The method of claim 21, wherein the first population of polymeric fibers, the second population of polymeric fibers, or both, comprises an anisotropic alignment of fibers.

29. The method of claim 28, wherein the first population of polymeric fibers, the second population of polymeric fibers, or both, comprises an arcuate alignment of fibers.

30. The method of claim 21, wherein at least some of the fibers in the first layer have a different alignment than some of the fibers of the second layer.

31. The method of claim 21, wherein at least some of the fibers in the second layer are oriented essentially perpendicular to at least some of the fibers of the first layer

32. A method, comprising:
electrospinning, from a polymeric fluid, a first population of polymeric fibers onto a first rotating surface of a mandrel,
the electrospinning being performed such that at least a portion of the first population of polymeric fibers is aligned on the first surface in an arcuate fashion.

33. The method of claim 32, wherein a spinneret containing the polymeric fluid is oriented essentially perpendicular to the plane of the first rotating surface of the mandrel.

34. The method of claim 32, wherein a spinneret containing the polymeric fluid is oriented essentially parallel to an axis about which the first rotating surface of the mandrel rotates.

35. The method of claim 32, wherein the polymeric fibers comprise a polymer that is natural, synthetic, biocompatible, biodegradable, non-biodegradable, biosorbable, or any combination thereof.

36. The method of claim 35, wherein the polymeric fibers comprise a population of nanoscale bodies.

37. The method of claim 32, further comprising depositing a cell onto the electrospun fiber.

38. The method of claim 32, wherein at least a portion of the first rotating surface has a linear velocity during electrospinning of between about 8 m/s and about 12 m/s.

39. The method of claim 32, further comprising electrospinning polymeric fiber so as to form a body having at least one cross-sectional dimension in the range of from about 10 micrometers to about 1 cm.

40. The method of claim 32, wherein the first rotating surface of the mandrel comprises a first conductive region and a second conductive region separated by an insulating region disposed there between.

41. The method of claim 40, wherein the electrospinning gives rise to a plurality of polymeric fibers aligned radially relative to an axis about which the first rotating surface of the mandrel rotates.

42. A composition, comprising:
a first layer comprising a first population of polymeric fibers,
the first population of polymeric fibers having an anisotropic alignment that varies spatially within the layer.

43. The composition of claim 42, wherein at least a portion of the first population of polymeric fibers has an arcuate alignment.

44. The composition of claim 42, wherein at least a portion of the first population of fibers has an average diameter in the range of from 10 nm to about 10 micrometers.

45. The composition of claim 42, further comprising a population of cells contacting the first population of fibers.

46. The composition of claim 42, wherein the first population of polymeric fibers comprises a biocompatible polymer.

47. The composition of claim 42, further comprising a second fibrous layer comprising a second population of fibers, the second fibrous layer being bonded to the first fibrous layer.

48. The composition of claim 47, wherein the second fibrous layer comprises a second population of fibers having an average diameter in the range of from 10 nm to about 10 micrometers.

49. The composition of claim 47, wherein the second population of fibers is aligned essentially parallel to one another.

50. The composition of claim 47, wherein the second population of fibers has an arcuate alignment.

51. The composition of claim 47, wherein the second population of fibers is aligned in a direction that differs from the alignment of the first population of fibers.

52. The composition of claim 42, wherein the first population of fibers comprises a population of nanoscale bodies disposed within.

53. The composition of claim 47, wherein the first layer, the second layer, or both, further comprises a porogenic material.

54. The composition according to claim 1, the composition being shaped to as to approximate at least a portion of a knee meniscus, an annulus fibrosis, or any combination thereof.

55. The composition according to claim 42, the composition being shaped to as to approximate at least a portion of a knee meniscus, an annulus fibrosis, or any combination thereof the laminate.
